(19) United States
(12) Patent Application Publication
(10) Pub. No.: US 2010/0324405 A1
(43) Pub. Date: Dec. 23, 2010

(54) ELECTRODE FOR ACQUIRING PHYSIOLOGICAL SIGNALS OF A RECIPIENT

(75) Inventors: Olli P. Niemi, Eindhoven (NL); Clive Van Heerden, Eindhoven (NL); Maria De Los Milagros Docamp Rama, Eindhoven (NL); Harald Reiter, Eindhoven (NL); Joerg Habetha, Eindhoven (NL); Ralf Schmidt, Eindhoven (NL); Matthew Harris, Eindhoven (NL); Guido J. Musch, Eindhoven (NL); Stijn H. W. Ossevoort, Eindhoven (NL); Oliver Gondorf, Eindhoven (NL); Sita A. C. Fischer, Eindhoven (NL)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N.V., Eindhoven (NL)

(21) Appl. No.: 12/670,455

(22) PCT Filed: Jul. 21, 2008
(86) PCT No.: PCT/IB08/52923

§ 371 Date: Jul. 26, 2010

(30) Foreign Application Priority Data
Jul. 26, 2007 (EP) 07113184.1

(51) Int. Cl. A61B 5/04

(52) U.S. Cl. 600/396

(57) ABSTRACT

The present invention relates to an electrode for acquiring physiological signals of a recipient. Furthermore the present invention relates to a textile fabric for use in a garment to be worn by a recipient, and to a monitoring system for monitoring of physiological parameters of a recipient. In order to provide an electrode for acquiring physiological signals of a recipient, which on the one hand provides a soft, and comfortable skin contact, whilst on the other hand assures a high signal quality, an electrode (1) for acquiring physiological signals of a recipient is suggested, which comprises at least two conductive textile layers (2, 3) positioned on top of each other, wherein the first layer (2) is made of a woven material, and the second layer (3) having a working surface (4) to be brought into contact with the recipient's skin is made of a knitted material.

### FIELD OF THE INVENTION

The present invention relates to an electrode for acquiring physiological signals of a recipient. Furthermore the present invention relates to a textile fabric for use in a garment to be worn by a recipient, and to a monitoring system for monitoring of physiological parameters of a recipient.

### BACKGROUND OF THE INVENTION

Monitoring systems for monitoring of physiological parameters of a recipient are known from the prior art. There are systems for monitoring heart rate, respiration and bio impedance, which can be used at home. Other systems, e.g. electroencephalographic (EEG) systems, electrocardiographic (ECG) or electromyographic (EMG) systems, are mainly adapted for clinical use.

In order to operate those systems, electrodes have to be used, which provide a skin contact to the recipient. To make such electrodes more user friendly and easy to use, e.g. in a home environment, textile electrodes has been suggested, which can be integrated into garments, e.g. into the recipients underwear.

During the last years, different kinds of textile electrodes have been developed. These are electrodes, which are directly applied to the recipient's skin without any use of a conductive jelly or the like. Because of the easy handling of such electrodes their user-comfort is clearly enhanced compared to wet electrodes mainly used in clinical applications. Experiments and testing has included a wide variety of different yarns and production techniques to achieve a reliable textile electrode for use in wearable garments for sensing physiological parameters of a recipient.

For example, knitted textiles have been used as electrode material for provide a comfortable skin contact. However, in weft knitted textile electrodes 30 the yarns 31 run in parallel across the surface area of the electrode, see FIG. 1. Thus, the resistance is significantly greater in one direction, which leads to a non-uniform resistance distribution across the electrode. If, for example, the yarns run horizontally, the conductivity fluctuates considerably in case of measuring horizontally. As a result, such knitted textile electrodes suffer from bad signal quality due high levels of noise disturbing the signal quality. In addition tests revealed, that a knitted electrode can retain moisture better than a woven electrode in order to reduce resistance.

On the other hand, electrodes 20 has been used with weave fabric as electrode material, providing a considerably more consistent and uniform resistance across the electrode area because of it's structure where the yarns 21 form a matrix, see FIG. 2. However, such textiles are not very user-friendly during skin contact. The woven textiles have been found out to be too abrasive and is prone to premature surface deformation.

### SUMMARY OF THE INVENTION

It is an object of the present invention to provide an electrode for acquiring physiological signals of a recipient, which on the one hand provides a soft, and comfortable skin contact, whilst on the other hand assures a high signal quality.

This object is achieved according to the invention by an electrode for acquiring physiological signals of a recipient, which electrode comprises at least two conductive textile layers positioned on top of each other, wherein the first layer is made of a woven material, and the second layer having a working surface to be brought into contact with the recipient's skin is made of a knitted material.

The object of the present invention is also achieved by a textile fabric for use in a garment to be worn by a recipient, which fabric is adapted to serve as a carrier for such an electrode.

The object of the present invention is also achieved by a monitoring system for monitoring of physiological parameters of a recipient, which comprises such an electrode and/or which comprises a garment with such a textile fabric.

A core idea of the invention is to provide a multi-layered conductive textile electrode for acquiring physiological signals, like heart rate, ECG signals or the like, with a knitted outer layer and a woven inner layer. The knitted outer layer provides a soft comfortable skin contact for reading biometric signals whilst the conductive woven layer underneath improves the conductive properties of knit structure. The matrix structure of the woven layer reduces and unifies the resistance across the surface area of the knitted skin contact layer, and reduces the noise levels. The reduced resistance and noise levels result in improved accuracy and consistency of the signal readings. This in turn reduces the complexity of the required monitoring software and enables an easier to realise and more reliable overall monitoring system.

Since the electrode according to the present invention is adapted for integration into textiles or garments the handling comfort is high and a monitoring system using such electrodes is suitable for long-term continuous monitoring of electrophysiological signals and other parameters. Because the electrodes suggested by the present invention are very robust, the monitoring system is more durable and reliable compared to prior art systems.

Depending on configuration and electronics the improved qualities allow the electrode to be used to detect heart rate, ECG, respiration and bio impedance or a combination of all of those, either in a hospital setting or at home. Compared with previous generations of textile sensors, the double layered conductive textile electrode provides a greatly improved performance in sensing biometric signals.

The present invention provides a user-friendly monitoring system with improved measurement quality. The invention is preferably applicable in the field of personal health care for continuously monitoring electro-physiological parameters at maximum comfort.

These and other aspects of the invention will be further elaborated on the basis of the following embodiments which are defined in the dependent claims.

According to a preferred embodiment of the invention at least one of the conductive textile layers are made by using a yarn, which comprises a conductive component and a non-conductive component. The components are in particular a metallic component and a textile component. The metallic component of such a metallic yarn is preferably stainless steel or silver. The selection of metal depends on the requirements of the specific application in terms of conductivity and robustness.

Compared to stainless steel silver is much more conductive. On the other hand, stainless steel is more robust than silver. The textile component is preferably a synthetic component, which is robust enough to serve as kernel for the metallic fibre. Preferably Polyester or Lycra are used as synthetic component, providing a robust fibre, which at the same time shows the elasticity and softness which is required for the described application.

Depending on the requirements of the specific application, not only the use of a single metallic component is possible (stainless steel/stainless steel; silver/silver). In a preferred embodiment a combination of both stainless steel and silver may be used, i.e. one of the conductive layers is made using a yarn, which comprises stainless steel as metallic component, and another conductive layer is made using a yarn, which comprises silver as metallic component (stainless steel/silver).

If stainless steel is used as metallic component, the yarn preferably comprises between about 20 and about 30 weight % stainless steel and between about 80 to about 70 weight % polyester. More preferably, the yarn comprises about 30 weight % stainless steel and about 70 weight % polyester. Using a yarn comprising more than 30 weight % of stainless steel would result in a very stiff and uncomfortable textile.

The use of a yarn as described above permits a long-term use without skin irritation and, at the same time, a good signal quality. A double layer electrode according to the present invention being manufactured using such a yarn was found to be comparable with a single layer woven electrode made of 100% silver.

The knitted layer may be weft knitted or warp knitted. Different techniques may also be applied for the woven layer. Preferably, industrial applicable knitting and weaving technologies are applied. The thickness of the layers can vary and depend on the requirements of the specific application.

In another preferred embodiment of the invention the electrode further comprises a support member, adapted to serve as a support for the conductive textile layers. The advantage of using such a support member is, that the layers can be positioned in a much more accurate way with respect to the recipient's skin, thus enabling a better signal to noise ratio.

For this purpose the support member preferably has a cushion-like shape, which enables the conductive textile layers to be stretched over the support member. By this means a very clean and uniform working surface can be obtained. This furthermore reduces the resistance of the knitted layer. Another advantage of the knitted layer being stretched is that stretching leads to a better conductivity of the knitted layer on its own. With the woven layer underneath the conductivity is significantly improved further.

Preferably, the support member is flexible. A main advantage of using a flexible support member is, that due to its flexibility it is assured, that the working surface of the electrode is in permanent contact to the recipient's skin, even if the wearer of the electrode moves. For this purpose the support member is preferably made of silicon, which is not only flexible, but also light-weighted, and cheap. However, other materials can be used for the support member instead, e.g. foam or any other material that is flexible, preferably non-toxicant, washable, and light-weighted.

The support member with the cushion-like shape is mainly responsible that the contact between textile electrode layer and skin is optimal while the person is moving. The support must be flexible and should guarantee that the conductive layer is all the time pressed constantly against the skin. Since movement artifacts caused by varying skin contact heavily disturb signal quality, the support member shows a preferred thickness of about 5 to about 10 mm, depending on where the electrodes will be placed on the person's body. For example a thickness of 10 mm is uncomfortable, if the electrode is applied in underpants, whereas in a shirt on the chest it is acceptable. The thickness is chosen such that an optimum compromise between signal quality and comfort is reached.

In a preferred embodiment of the invention the electrode is attached to a textile fabric or cloth, which is intended to be used in a garment to be worn by a recipient. The fabric serves as a carrier for the electrode. For this purpose the fabric preferably comprises an opening adapted to fit the size of the working surface of the second layer of the electrode. In other words, the electrode is inserted through the opening and subsequently connected to the fabric.

Preferably, the support member of the electrode is positioned to the fabric in a way, that results in a raised profile, which provides a better skin contact when the electrode is integrated into the fabric. Again, this improves the signal quality of the electrode.

Preferably the layers of the electrode are connected to the fabric by lockstitch sewing or by heat bonding, preferably by ultra sonic welding. These industrial processes are quick, robust, and keeping the overall production costs low.

Preferably, the fabric is made of a non-stretchable material to ensure optimum contact between the layers and to maintain a constant surface area. Thus, once the electrode is connected to the fabric, it cannot unintentionally slip or shift, which leads to more accurate signal readings.

### BRIEF DESCRIPTION OF THE DRAWINGS

These and other aspects of the invention will be described in detail hereinafter, by way of example, with reference to the following embodiments and the accompanying drawings; in which:

FIG. 1 shows a schematic illustration of a weft knit structure,

FIG. 2 shows a schematic illustration of a plain weave structure,

FIG. 3 shows a top view of an electrode integrated into a fabric, and

FIG. 4 shows a cross sectional view of the electrode along section IV-IV.

### DETAILED DESCRIPTION OF THE EMBODIMENTS

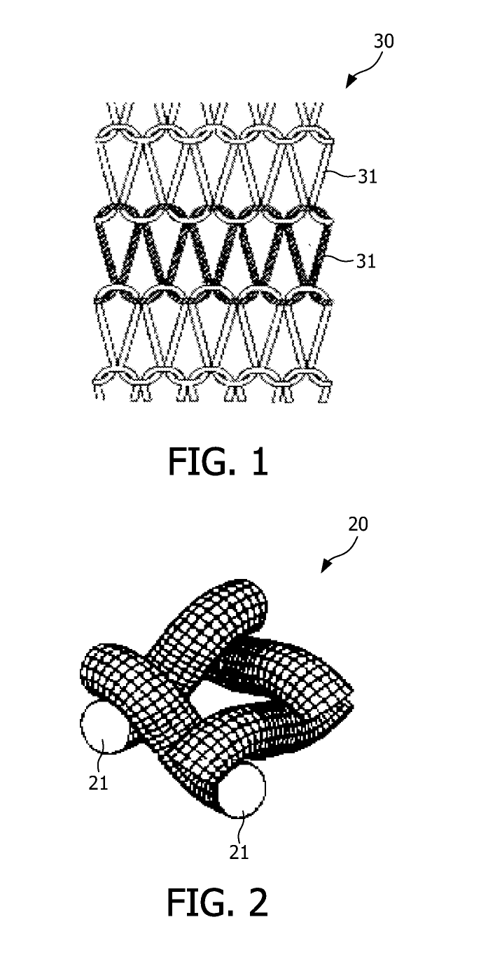

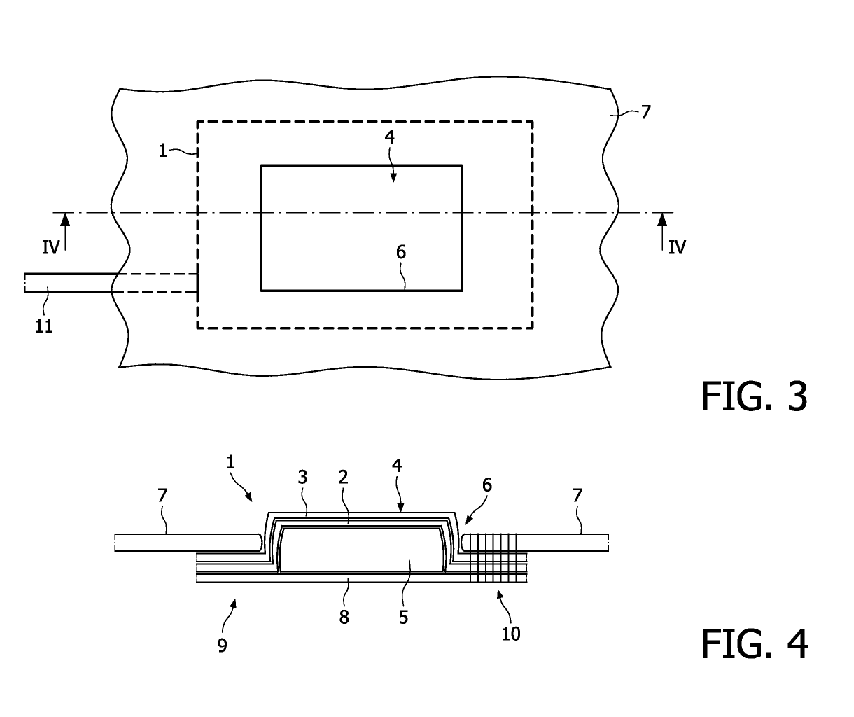

The electrode 1 according to the present invention is adapted for acquiring physiological signals of a recipient, which might be used in a ECG monitoring system (not shown). The electrode 1 comprises two conductive textile layers 2, 3 positioned on top of each other, wherein the first layer 2 is made of a woven material (see FIG. 2), and the second layer 3 having a working surface 4 to be brought into contact with the recipient's skin (not shown) is made of a knitted material (see FIG. 1). The first layer 2 ensures a soft skin contact and improved wearability and the second layer 3 makes the conductivity uniform all across the working surface 4 of the electrode 1.

Both conductive textile layers 2, 3 are made by using a yarn, which is comprised of stainless steel and polyester. The yarn comprises about 30% stainless steel and about 70% polyester.

The two layers 2, 3 are stretched or pulled over a flexible silicone cushion 5, which serves as a support member. The layers 2, 3 and the cushion 5 are inserted through an opening 6 in a textile fabric 7, which opening is adapted to fit the size of the working surface 4 of the second layer 3. Thereby the electrode 1 is positioned in the opening 6 of the fabric 7 such that the working surface 4 is accessible. The textile fabric 7 is made of a non-stretchable material, e.g. cotton. In order to maintain the cushion 5 in its end position, an additional covering element 8 is provided, covering the bottom side of the cushion 5 and extending towards the edges of the layers 2, 3. The covering element 8 may be a piece of a preferably non-conductive textile fabric, for example.

Subsequently the border areas 9 of the layers 2, 3 around the cushion 5 and the extended edges of the covering element 8 are sewn to the fabric 7 by a number of stitches 10. In FIG. 4 only stitches at one side of the electrode 1 are shown. Preferably, the layers 2, 3 and the covering element 8 are seamed along their outer borders to prevent the layers 2, 3 to fray. The height of the cushion 5 is chosen according to the thickness of the fabric 7 such that a permanent contact between the working surface 4 and the skin is guaranteed. In the illustrated end position the cushion 5 protrudes the fabric 7, which leads to a raised position of the working surface 4.

In the border area 9 of the layers 2, 3 adjacent the opening 6 a cable 11 is connected to the electrode 1. The cable 11 is connected at the left or right margin of the conductive layers 2, 3, preferably using lockstitch sewing, heat bonding (e.g. ultra sonic welding), or another suitable technology. Preferably both layers 2, 3 are connected to the cable 11. The cable 11 is preferably made of a conductive textile. The cable 11 connects the electrode 1 with electronics (not shown) of the monitoring system. Instead of the connecting cable 11 a radio frequency transmitter device may be used for wireless data transmission to an external monitoring device.

The fabric 7 as a carrier for the electrode 1 is then used in a garment or as part of a garment to be worn by a recipient. Electrode 1 and garment can be produced and assembled separately. The including of the electrode 1 does not limit the garment design. The electrode 1 can be fixed to an arbitrary position of the garment. Because of the reduced number of production steps and the reduced production costs compared to other attachment methods the electrodes 1 are suitable for mass production.

The electrode 1 according to the present invention is preferably designed to be integrated into wearable bio sensing garments improving consumer comfort while providing reliable independent monitoring. It can be incorporated into medical, health and wellness and sports applications ranging from relatively easy to execute heart rate monitor to more complete and complex bio sensory systems.

It will be evident to those skilled in the art that the invention is not limited to the details of the foregoing illustrative embodiments, and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. It will furthermore be evident that the word “comprising” does not exclude other elements or steps, that the words “a” or “an” do not exclude a plurality, and that a single element, such as a computer system or another unit may fulfil the functions of several means recited in the claims. Any reference signs in the claims shall not be construed as limiting the claim concerned.

### REFERENCE NUMERALS

1. electrode

2. first layer

3. second layer

4. working surface

5. cushion

6. opening

7. fabric

8. covering element

9. border area

10. stitch

11. cable

20. knitted fabric

21. yarn

30. woven fabric

31. yarn

1. An electrode (1) for acquiring physiological signals of a recipient, said electrode (1) comprising at least two conductive textile layers (2, 3) positioned on top of each other, wherein the first layer (2) is made of a woven material, and the second layer (3) is made of a knitted material having a working surface to be brought into contact with the recipient's skin.

2. The electrode (1) as claimed in claim 1, wherein at least one of the conductive textile layers (2, 3) are made by using a yarn, which comprises a metallic component and a synthetic component.

3. The electrode (1) as claimed in claim 2, wherein the metallic component is stainless steel or silver and the synthetic component is polyester.

4. The electrode (1) as claimed in claim 3, wherein the yarn is comprised of about 20 to about 30 weight % stainless steel and about 80 to about 70 weight % polyester.

5. The electrode (1) as claimed in claim 1, further comprising a support member (5), adapted to serve as a support for the conductive textile layers (2, 3).

6. The electrode (1) as claimed in claim 5, wherein the support member (5) has a cushion-like shape.

7. The electrode (1) as claimed in claim 5, wherein the conductive textile layers (2, 3) are stretched over the support member (5).

8. The electrode (1) as claimed in claim 5, wherein the support member (5) is flexible.

9. A textile fabric (7) for use in a garment to be worn by a recipient, said fabric (7) being adapted to serve as a carrier for an electrode (1) as claimed in claim 1.

10. The textile fabric (7) as claimed in claim 9, comprising an opening (8), said opening (8) being adapted to fit the size of the working surface (4) of the electrode (1).

11. The textile fabric (7) as claimed in claim 9, to which the electrode (1) is connected either by lockstitch sewing or by heat bonding.

12. The textile fabric (7) as claimed in claim 9, made of a non-stretchable material.

13. A monitoring system for monitoring of physiological parameters of a recipient, comprising an electrode (1) as claimed in claim 1.

14. A monitoring system for monitoring of physiological parameters of a recipient, comprising a garment comprising a textile fabric as claimed in claim 9.
